(19) United States
(12) Patent Application Publication
(10) Pub. No.: US 2023/0187088 A1
(43) Pub. Date: Jun. 15, 2023

(54) SYSTEM AND METHOD FOR IMPROVED DENTISTRY

(71) Applicant: Scuyler Myron Robert Kurlbaum, Leawood, KS (US)

(72) Inventor: Scuyler Myron Robert Kurlbaum, Leawood, KS (US)

(21) Appl. No.: 18/065,724

(22) Filed: Dec. 14, 2022

Related U.S. Application Data
(60) Provisional application No. 63/265,367, filed on Dec. 14, 2021.

(51) Int. Cl. G16H 80/00; G16H 20/40; G16H 30/20; G16H 10/60

(52) U.S. Cl. CPC G16H 80/00; G16H 10/60; G16H 20/40; G16H 30/20

(57) ABSTRACT

A system and method for improved dentistry comprises an information and technology system to allow a dentist or group of dentists to participate in and review the collection of patient information while remote from the location of the patient. Data and information from CAD, x-ray, image, and measurement capture devices is acquired from patients. Upon review of the data and recommendation by the dentist, appliances or devices may be manufactured using CAM systems, and the dentistry is completed at a physical location by performing a physical treatment or procedure on the patient. The system and method allow individual dentists, dental practice groups, and DSOs to maximize their efficiency and capacity to increase patient care and to minimize costs.

### CROSS REFERENCE TO RELATED APPLICATIONS

This application claims priority to U.S. Provisional Application Serial No. 63/265,367, filed Dec. 14, 2021, which is hereby incorporated herein in its entirety by reference.

### BACKGROUND OF THE INVENTION

Modern dental care and dentistry procedures such as teeth cleanings, filings, extractions, and crowns all rely on an integration of technology with the knowledge, expertise, and hands-on performance of the various procedures by a dentist. For example, most dental offices rely on x-rays or scanners to obtain images of patients’ teeth, as well as using other measurement devices to obtain various patient data, with all of the gathered data typically stored in a local computer system that itself is often integrated into a larger network of computers. A typical dentist office may also include multiple stations for accommodating numerous patients at once, or for providing specialized equipment at a particular station.

In addition to the more common procedures of cleaning and fillings, dentists may further offer cosmetic and restorative procedures such as crowns, veneers, teeth whitening, root canals, dentures, and orthodontia such as braces, aligners, and retainers.

Regardless of the procedure, a dentist typically must monitor or provide supervision for each procedure performed by hygienists or other assistants, and additionally must personally perform many of the procedures. Thus, the day-to-day operation of a dentist office revolves around the involvement and participation of the dentist with the patients and the treatments and procedures being performed.

In the event a dentist is out of the office, most procedures on patients cannot be performed - and those that are performed by hygienists or assistants must still be reviewed by the dentist when he or she returns to the office. And, while tele-dentistry (i.e., communication with a dentist via a mobile communications device) is known and used, in most cases it is an ad-hoc solution to the dentist being out of the office unexpectedly.

Thus, it can be seen that there remains a need in the art for a system and method that allows dentists, dental offices and dental services offices (DSOs) to more efficiently provide services for the benefit of both the patients and the dentists.

### SUMMARY OF THE INVENTION

Embodiments of the invention are defined by the claims below, not this summary. A high-level overview of various aspects of the invention is provided here to introduce a selection of concepts that are further described in the detailed description section below. This summary is not intended to identify key features or essential features of the claimed subject matter, nor is it intended to be used in isolation to determine the scope of the claimed subject matter.

The present invention is directed to systems and methods for improved dentistry that integrate dental technology and communications technology to allow dentists, individual dental offices, and dental services offices (DSOs) to more efficiently and effectively provide dental services to patients.

In one exemplary embodiment, dental technologies such as computer aided design (CAD) and computer aided manufacturing (CAM) technologies, such as dental scanning technologies, are employed by hygienists, dental assistants, dental therapists, dentists, non-dentists, patients, or others to collect images and/or information and data relating to patients’ dental care. Additional information, such as X-rays, images, and measurement information, are also collected. For example, photos, camera images, videos, X-ray images, impressions, measurements, and other information is gathered using the corresponding technologies and devices. The collected data is provided to a supervising dentist either synchronously - i.e., in real time, with the dentist participating in-person or via remote communication contemporaneously with the data and information collection, e.g., tele dentistry - or asynchronously, wherein the dentist reviews the collected data and information at later time.

With the data and information gathered and reviewed by a dentist, the dentist recommends proposed treatments or procedures, which may include common or routine procedures, such as fillings; cosmetic treatments such as teeth whitening or veneers; restorative procedures such as root canals, crowns, or dentures; orthodontia such as braces, aligners, and retainers; or other treatments and/or procedures as recommended by the dentist.

Once the recommended treatment(s) and procedure(s) are discussed with the patient and a course of treatment is agreed upon, the patient receives the treatment(s) and procedure(s) from the dentist at a physical dental office, which may be the same office at which the CAD, X-ray, image, impression, and measurement data was gathered (if the data was gathered at the office); may be at a different physical office location; or may be at a centralized office that accommodates multiple patients from one or more dentists. If a restorative device, such as a crown or veneers, or an appliance, such as a retainer or aligner, are required, the collected CAD and other measurement data may be sent to a computer aided manufacturing (CAM) device for manufacture of the device or appliance. The CAM device may be a single machine configured to manufacture at least one device or appliance, or may be a facility or laboratory that processes the data and prepares the device according to the required parameters.

The integration of the data and information gathering process with the remote communication and participation of a dentist allows dental offices or DSOs to more efficiently and effectively use and schedule their dentists and other employees and to provide more efficient and effective treatment of patients or administration of procedures

Thus, rather than employing tele-dentistry on an ad-hoc basis, tele-dentistry is integrated within the entire process and within the technology used in conjunction with the overall practice management. While effective to increase the efficiency of a single dentist office, the system and method of the present invention are likewise well-suited to accommodate large dental practices and DSOs having multiple locations and multiple dentists.

Patients are able to have initial consultations and examinations at convenient locations and times, with the data and information gathered being instantly available to an on-site or remote dentist, under the dentist’s supervision. The actual treatments and procedures are performed in physical dental offices by the dentist that provided the initial assessment, or in alternative embodiments, by a different dentist.

### BRIEF DESCRIPTION OF THE DRAWINGS

A more complete understanding of the present invention is preferably obtained by reference to the detailed description when taken in conjunction with the accompanying drawings wherein:

FIG. 1 is a flow diagram of a method for improved dentistry in accordance with an exemplary embodiment of the present invention.

FIG. 2 is a detailed view of a portion of the flow diagram of FIG. 1.

FIG. 3 is a detailed view of a portion of the flow diagram of FIG. 1.

FIG. 4 is a detailed view of a portion of the flow diagram of FIG. 1.

FIG. 5 is a detailed view of a portion of the flow diagram of FIG. 1.

FIG. 6 is a detailed view of a portion of the flow diagram of FIG. 1

FIG. 7 is a detailed view of a portion of the flow diagram of FIG. 1.

FIG. 8 is a schematic diagram of a system for improved dentistry in accordance with an exemplary embodiment of the present invention.

### DETAILED DESCRIPTION

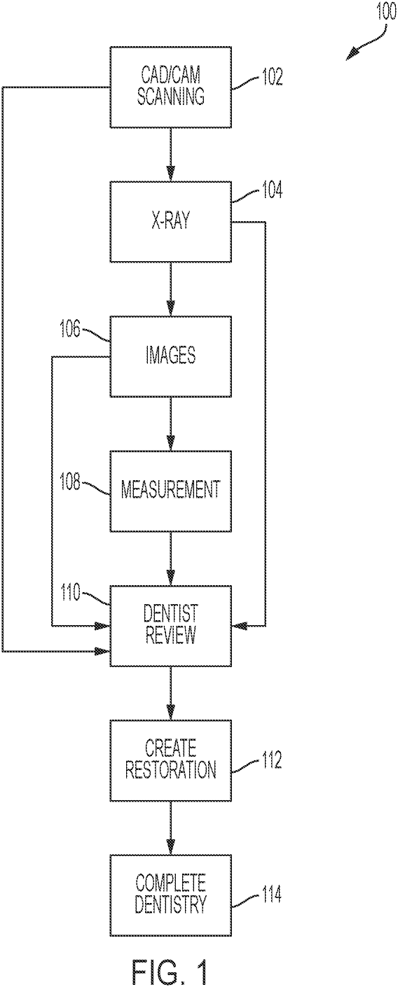

Looking to FIGS. 1 through 8, a system and method of improved dentistry in accordance with exemplary embodiments of the present invention are depicted in flow and block diagram form. It should be understood that variations from the described exemplary embodiments are within the scope of the present invention. For example, the system and method may exclude some features described, may combine the described features in configurations other than those specifically described, and may include additional features not described. These and other variations are contemplated by the present invention.

Looking first to FIG. 1, a flow-diagram of an exemplary method for improved dentistry is depicted generally by the numeral 100. At block 102 intraoral computer aided design (CAD) scanning of a patient’s tooth, teeth, or mouth is performed using a three-dimensional (3D) CAD scanning device to capture 3D image data of the desired portions of the oral cavity. The captured 3D images are preferably in a format compatible with related computer aided manufacturing (CAM) devices, such as molding machines, 3D printers, milling machines, and other CAM devices used to construct dental devices and/or appliances. The captured scans and data are preferably stored in a computer, database, or practice management system for eventual use by a CAM device and for review by a dentist. It should be understood that all procedures may be perfumed under the direct supervision of a dentist, or that supervision by the dentist may occur at any point in the process, such as during the review of the capture data.

At block 104, X-ray data may be collected, typically in the form of two-dimensional film camera images. The captured X-ray images are likewise preferably stored in a computer, database, or practice management system for review by a dentist. At block 106 other image data, such as photographs, may be captured with the images stored for further review.

At block 108 other measurement data, such as gum, plaque, decay, and other measurements captured by micrometers, picks, impressions, or other dental tools and data captured in the course of a dental examination, teeth cleaning, or other procedures is captured and stored in the computer, database, or practice management system for archival purposes and for later review. Other devices or specialized dental machines may be used to capture additional information or other types of information.

It should be understood that all of the collected data is preferably digitized and stored on the computer, database, or practice management system. And, as will be described in more detail below, the digitized data is accessible by remote dentists, dentist offices, and DSO’s, allowing retrieval and review of the data either synchronously - i.e., with a dentist participating in the capture of the data either by being physically present while the data is captured or by being virtually present via a remote communications device - or asynchronously, with the captured data reviewed by a dentist at a later time, either in the office in which the data was collected, or remotely from a different office or location, or at a DSO or office where the treatment or procedure is performed.

It should be further understood that the steps of blocks 102, 104, 106, and 108 are exemplary and not limiting of the scope of the present invention, and that implementation of the method of the present invention may not require all of the steps and data acquisition as just described. For example, in some cases CAD/CAM scanning 102 may be the only data/information acquired. In other cases, only images 106 and/or x-rays 104 may be obtained. In other cases, all four types of information 102, 104, 106, and 108 may be required. Depending on the patient’s needs and the information required by the dentist, some of the steps may be not be necessary in that particular case. It should be further understood that the order of the steps may be other than that depicted in the exemplary embodiment of FIG. 1 - for example, images 106 and x-rays 104 may be taken after gathering other information in block 108. These and other variations are contemplated by the present invention.

With the various data acquired, at block 110 a dentist reviews the collected data, images, and information in the course of recommending a treatment or procedure for the patient. For example, the dentist may recommend a filling, crown, or other procedure, or may recommend aligners, braces, orthodontia, or other appliance or device depending on the needs of the patient.

Looking still to FIG. 1, it should be understood that the dentist review 110 may occur concurrently with any or all of the data collection steps 102, 104, 106, and 108. For example, in a small dental office, the dentist may be present with the patient during those data collection steps. In a preferred embodiment, the dentist may participate synchronously via a telecommunications device such as a smartphone, tablet, and the like during the data collection steps. Thus, the dentist may be located remotely while directing and observing the collection of patient data, and may be reviewing 110 that data as the data collection is taking place. Thus, as seen in FIG. 1, each of the scan, X-ray, data collection, and image steps 102, 104, 106, and 108 is tied directly to the dentist review step 110 indicating that dentist review can occur essentially simultaneously with those steps. And, as described previously, not all of the steps of information collection may be necessary in every case.

As just described, it can be seen that a dentist can participate in and review the collection of patient information while remote from the location of the patient. Thus, a dentist with a single office can see patients (via a remote communications device) and review collected data and information just as if he or she was in the office with the patient. Furthermore, the integration of the data collection, review, and tele dentistry/communication technology as just described allows a dentist to see patients at different locations - e.g., at different offices - without requiring the dentist to physically travel to those different locations. And, in the case of dental services offices (DSOs) having multiple locations each with the capability to see several patients at once, multiple dentists may see multiple patients a day, even at different offices, with the dentists located remotely and not required to go to all - or any - of the offices. Thus, the method of the present invention allows individual dentists, dental practice groups, and DSOs to maximize their efficiency and capacity to increase patient care and to minimize costs in achieving that capacity.

Regardless of whether the data and information collection was performed synchronously or asynchronously, at block 112 any required restoration, device, or appliance may be created using the collected data. For example, the collected CAD data and other measurement, X-ray, and image data may be sent to a computer aided manufacturing (CAM) facility or machine, such as a milling machine, 3D printer, or molding machine to create the required device or appliance.

Finally, at block 114, the method is completed with the dentist treating the patient and/or completing the physical procedure, such as filling a tooth, placing a crown, installing orthodontia, or any other treatment or procedure as called for by the dentist based on his or her review 110 of the patient information. In a preferred embodiment, the dentistry is performed by the same dentist who reviewed the patient information and recommended the treatment or procedure, in other embodiments the dentistry may be performed by a dentist other than the original dentist, and in further embodiments, the dentistry may be performed at another office or DSO remote from the office in which the data was collected.

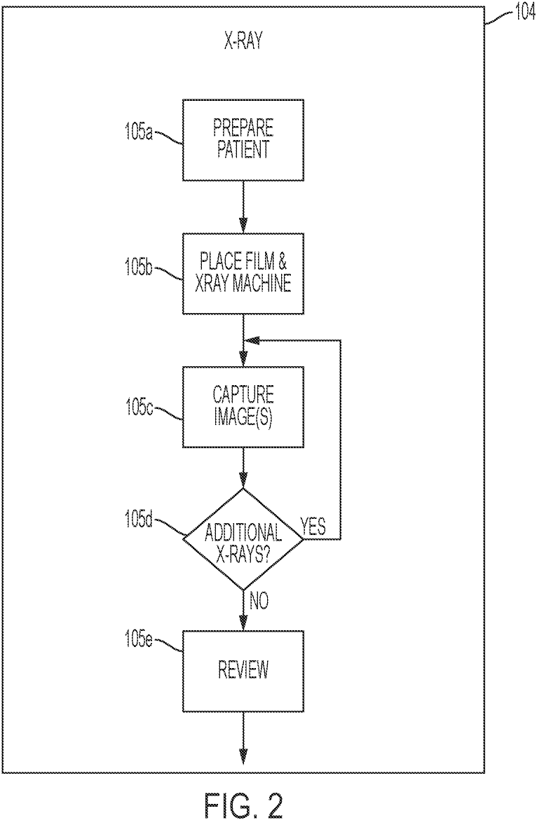

With the method of improved dentistry generally set forth in FIG. 1 and described above, FIGS. 2 through 7 provided a more detailed flow diagram view of the CAD/CAM scanning of block 102, the X-ray capture of block 104, the image capture of block 106, the measurement capture of block 108, and the creation of a restoration of block 112.

Looking to FIG. 2, a detailed view of the X-ray block 104 of FIG. 1 shows that at block 105a, a patient is prepared for taking X-rays. Preparation generally includes positioning the patient and placing protective equipment and coverings, such as a lead lined cloak. At block 105b, film is placed adjacent the tooth or area to be captured, and the X-ray machine is positioned in proximity to the film. At block 105c, X-ray images are captured by activating the X-ray machine, with an image captured on the film. At decision block 105d, if additional images are required, then the process of block 105c is repeated at the new location. If no additional X-rays are required, at block 105e, the captured data is reviewed by the technician performing the X-rays to ensure the images have been properly captured.

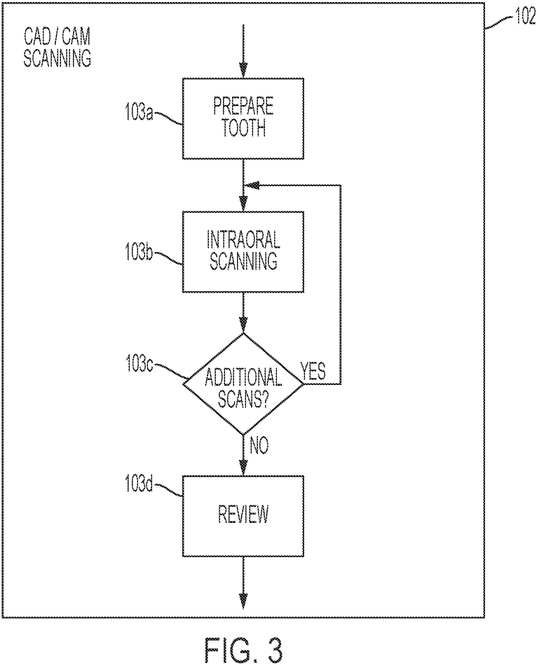

Turning to FIG. 3, a detailed view of the CAD/CAM Scanning block 102 of X-ray block 104 of FIG. 1 shows that at block 103a, a tooth (or other intraoral region) is prepared for scanning, with the scanning device positioned to capture the desired tooth or area. At block 103b, the scanning is performed. At decision block 103c, if other scans are required, the CAD scanning process of block 103b is repeated. If no additional CAD scanning is required, at block 103d the captured data is reviewed by the technician performing the scans to ensure that the images have been properly captured.

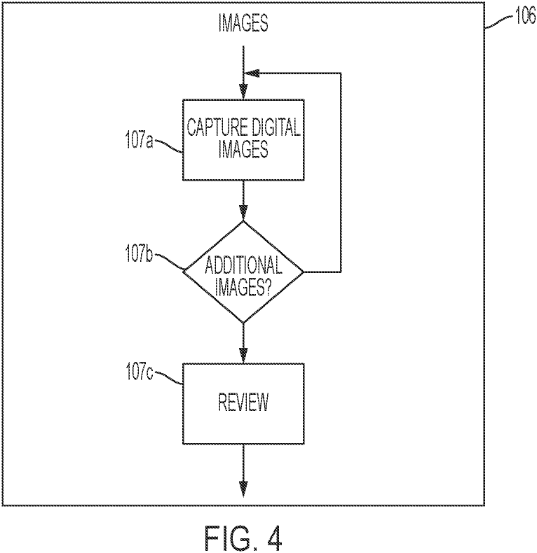

As seen in FIG. 4, a detailed view of the Images block 106 of FIG. 1 shows that at block 107a digital images of a tooth, area, or other region are captured. At block 107b, if other images are required, the image capturing process of block 107a is repeated. If no additional image capture is required, at block 107c the captured data is reviewed by the technician taking the digital photos to ensure that the images have been properly captured.

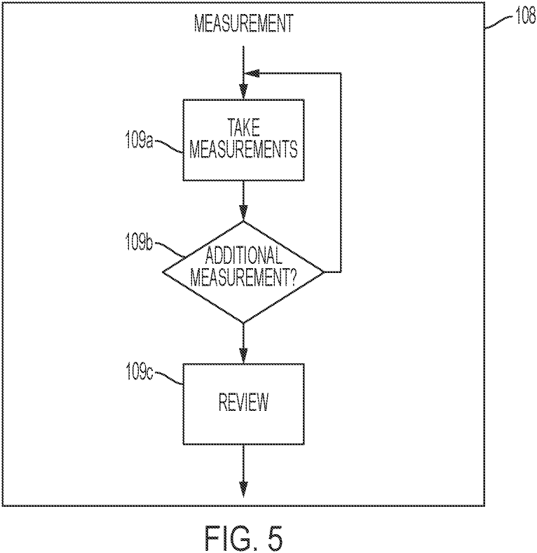

In FIG. 5, a detailed view of Measurement block 108 of FIG. 1 shows that at block 109a, measurements are captured of a tooth, area, or other intraoral region. For example, a technician or hygienist may capture gum, plaque, or decay measurements and record the measurements, preferably in the computer, database, and/or office management system. At decision block 109b, if no additional measurements are required, at block 109c , recorded data is reviewed by the technician performing and entered into the computer system.

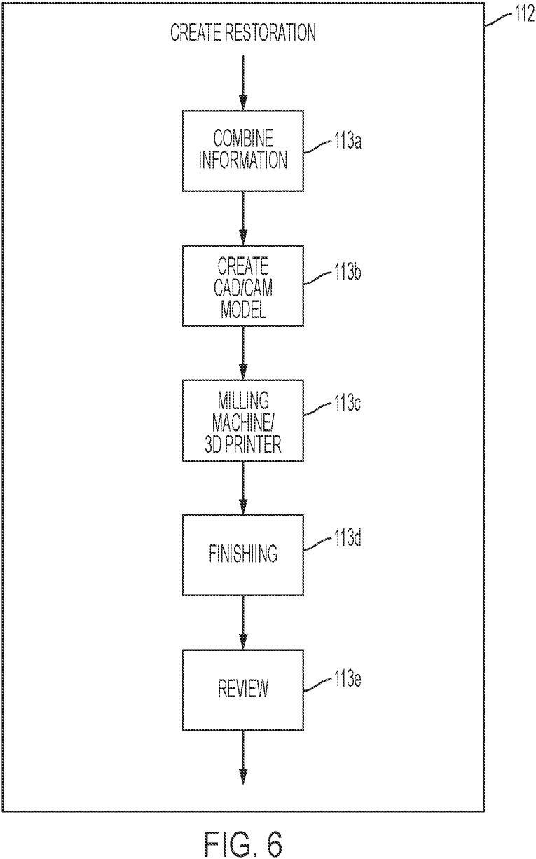

FIG. 6 depicts a detailed view of the Create Restoration block 112 of FIG. 1. At block 113a, all of the captured information (CAD/CAM data, X-ray data, Image data, Measurement data, and any other desired data or information) is aggregated or collated to create a CAD/CAM model at block 113b. At block 113c, a desired device or appliance is created using the CAD/CAM model. For example, the CAD/CAM model may be transmitted to a milling machine, molding machine, 3D printer, or other machine for manufacturing the device or appliance. At block 113d final finishing of the manufactured device or appliance is performed if required, and at block 113e, the device or appliance is inspected to ensure that it conforms to the desired model.

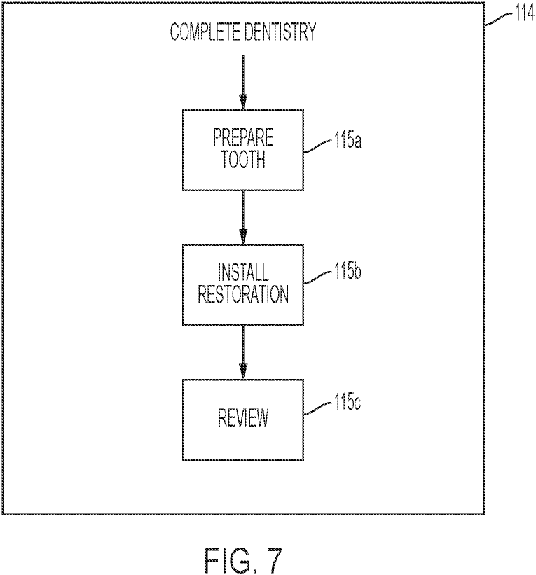

Looking to FIG. 7, a detailed view of the Complete Dentistry block 114 of FIG. 1 shows that at 115a the tooth (or other intraoral region) is prepared to accept the created appliance or device, and at block 115b, the device or appliance is installed or fitted as required. At block 115c, the installation or fitment is reviewed and confirmed. It should be understood that the steps of FIG. 7, and the entire Complete Dentistry of block 114 are preferably performed by a dentist at a physical office or DSO location.

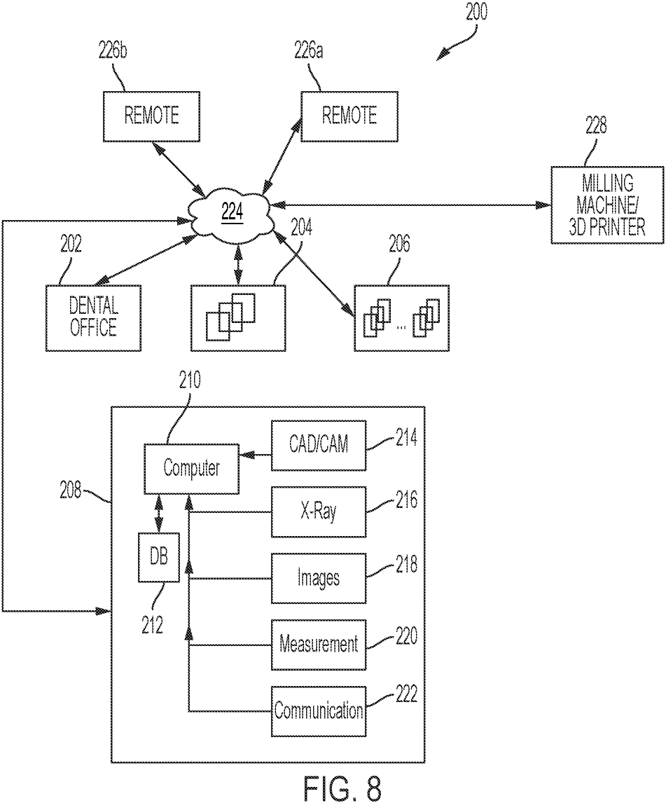

Turning to FIG. 8, a system for performing the method as just described is depicted generally by the numeral 200. The system 200 comprises one or more individual dentist offices 202, affiliated dentist offices 204, and dental services offices (DSOs) 206, or combinations thereof. An individual dentist office is a typical single or multiple dental practice having equipment and technology such as that depicted in block 208, comprising a computer 210, a database 212, CAD/CAM scanning equipment 214, x-ray equipment 216, imaging equipment 218, and other measurement equipment 220. The equipment and technology of block 208 is preferably configured as a remote system or a remotely accessible system. As described previously, each piece of measurement equipment 214, 216, 218, 220 collects various data and information from a patient and aggregates that information and data into the computer 210 and database 212 system for archival and review purposes. It should be understood that information may be transmitted from the equipment to the computer/database electronically (e.g., an image file may be directly transferred) or may be manually entered by a dental assistant or may be otherwise entered into the system.

The equipment and technology system 208 preferably further includes a communication device or means 222, such as a mobile communications device, smart phone, tablet, or other telecommunications device preferably having communications circuitry capable of transmitting and receiving image and voice information to allow a dentist to remotely participate in the patient examination process. Most preferably, the communication device 222 is configured to transmit and receive information to and from the computer system and database and/or to and from the individual data collection equipment to allow a remotely located dentist to review and observe collected data. The equipment and technology system 208 is connected to a network 224 to allow the equipment and technology at a particular dentist office or location to communicate to other equipment and technology at other locations, and to communicate with one or more dentists or other users connected remotely through the network via remote communications devices 226a, 226b. Network 224 may comprise local area networks (LANs), wide area networks (WANs) such as the Internet, or combinations thereof.

Each of the dentist offices 202, affiliated dentist offices 204, and dental services offices (DSOs) 206 preferably comprise one or more equipment and technology systems 208 operable to communicate with other offices within the affiliated offices or DSOs, and with dentists connected remotely via communications devices 226a, 226b.

Computer aided manufacturing (CAM) machines or facilities 228 are connected to the network 224 to allow CAD/CAM model data to be transmitted to the device of facility for manufacture of devices, appliances, molds, or other desired pieces as described above.

Looking to FIGS. 1 through 8 in conjunction, and with reference to the method of FIGS. 1 through 7 as previously described, the system and method of the present invention allow a dentist, or dentists, to participate in and review the collection of patient information while remote from the location of the patient. The integrated data collection, review, and tele-dentistry and communication technology allows a dentist to see patients at different locations - e.g., at different offices - without requiring the dentist to physically travel to those different locations.

Furthermore, they system and method of the present invention allow dental services offices (DSOs) having multiple locations each with the capability to see several patients at once, multiple dentists may see multiple patients a day, even at different offices, with the dentists located remotely and not required to go to all - or any - of the offices.

And, while the system and method of the present invention provide for improved dentistry incorporating and leveraging dental and communications technology, as described previously with respect to block 110 of FIG. 1, all dentistry is completed in-person with a dentist and a physical treatment or procedure is performed in accordance with the recommendations of the dentist achieved through use of the system and method.

It should be understood that the operation of the system and method of the present invention are exemplary in nature and not limiting, and that the various operations may proceed in the sequence depicted, or in any other sequences, as would be apparent to those of skill in the art.

1. A method for improved dentistry, comprising:
capturing patient data related to a treatment or procedure for a patient;
storing the captured patient data on a remotely accessible system;
accessing the remotely accessible system and reviewing the stored patient data;
recommending a treatment or procedure for the patient; and
performing a physical procedure or treatment on the patient.

2. The method of claim 1, wherein capturing patient data related to a treatment or procedure includes capturing: CAD/CAM data, x-ray data, image data, measurement data, or combinations thereof.

3. The method of claim 1, wherein the remotely accessible system comprises a computer, a database and communication circuitry and wherein the remotely accessible system is in communication with a network.

4. The method of claim 3, wherein accessing the remotely accessible system comprises communicating with the remotely accessible system via a mobile communication device.

5. The method of claim 1, further comprising creating a physical appliance or device for use in treating the patient.

6. The method of claim 5, wherein creating a physical appliance or device comprises transmitting CAD/CAM data to a CAM device.

7. The method of claim 5, wherein performing a physical procedure or treatment comprises installing the created physical device.

8. The method of claim 5, wherein the created physical device comprises veneers or aligners.

9. The method of claim 1, wherein capturing patient data comprises allowing supervision by a dentist either synchronously or asynchronously.

10. A system for improved dentistry, comprising:
at least one dental device operable to capture information and data related to treatments or procedures for a patient;
a communications device operable to allow a dentist located remotely from the patient to participate in collecting and reviewing patient data; and
a physical dentistry location configured to allow a dentist to complete recommended dentistry on the patient.

11. The system of claim 10, wherein the at least one dental device comprises a CAD/CAM measurement device, an x-ray machine, a camera operable to capture image data, a micrometer, a pick, or combinations thereof.

12. The system of claim 10, wherein the communications device comprises a mobile device.

13. The system of claim 10, further comprising a computer and database in communication with a network.

14. The system of claim 13, further comprising a CAM machine or facility in communication with the network.

15. The system of claim 13, wherein the computer and database are located at a dentist office, a DSO, or combinations thereof.

16. The system of claim 10, wherein the collected information and data is aggregated and stored for remote access.

17. The system of claim 10, further comprising a CAM device operable to create a device or appliance corresponding to the collected information and data.

18. The system of claim 17, wherein the CAM device is in communication with a database or computer storing the collected information and data.
